(19) United States
(12) Patent Application Publication
(10) Pub. No.: US 2010/0010318 A1
(43) Pub. Date: Jan. 14, 2010

(54) Identifying Products Containing a Food Item That Cause a Food Sensitivity

(75) Inventor: JENS RICHTER, Magdeburg (DE)

Correspondence Address: Buchanan Ingersoll & Rooney PC (SEN), 301 Grant Street, 20th Floor, Pittsburgh, PA, 15219, US

(73) Assignee: SIEMENS ENTERPRISE COMMUNICATIONS GMBH & CO. KG, Munchen (DE)

(21) Appl. No.: 12/171,324

(22) Filed: Jul. 11, 2008

(51) Int. Cl. A61B 5/00; G06K 7/10; G09B 19/00

(52) U.S. Cl. 600/300; 434/127; 128/920; 235/462.42; 235/462.45

(57) ABSTRACT

In one aspect a mobile health monitor that indicates that a food product is suitable for a sufferer of a food sensitivity is provided. The mobile health monitor includes a reader that reads the barcode of the food product, a database having information about a plurality of food products to determine the suitability of the respective food product, and an indicator that provides an indication when a food product is suitable for the suffer. The database is searched base on the read barcode to determine if the food product associated with the barcode is suitable. The indicator indicates that the food product is suitable when the food product is determined suitable based on the searched database. A method indicating that a food product is suitable for a sufferer of a food sensitivity is also provided.

### FIELD OF INVENTION

The present invention relates to the identification of a food item in a food product.

### BACKGROUND OF INVENTION

Over the past few years there has been an increase in the number of people who have sensitivity to a particular food such that the World Health Organization (WHO) regards food allergies as the one of the world's largest public health problem. The term “food sensitivity” is used herein to describe any sensitivity to a particular food when ingested such as food allergy or food intolerance. A study in France involving 33,110 people indicated that about 3.24% of the population is likely to have a food allergy. A study in the UK indicated that 45% had a food sensitivity with about 2% suffering from a food allergy.

Symptoms for food sensitivity may manifest themselves in many different ways. For example, areas affected may include the mouth, upper/lower respiratory tracts, digestive track, skin, and circulatory system. Mouth area complaints may include furry felling on the tongue, itchy throat and swollen lips. Respiratory tract problems may include itchy eyes, runny nose, and difficult in breathing. Digestive track problems may include flatulence, diarrhea, stomach ache and vomiting. Skin problems may include hives and a worsening of a pre-existing atopic eczema. Circulatory reactions may include allergic shock with difficulty in breathing, loss of consciousness and even cardiovascular cessation.

Treatment of a food sensitivity first involves identifying the food item that causes the sensitivity. The most common food items that cause sensitivity are milk, egg protein, wheat, soy, nuts, legume, fish, spices, and gluten. Once the food item is identified the individual must no longer consume the food product having the food item.

### SUMMARY OF INVENTION

In one aspect, a mobile health monitor is provided that indicates that a food product is suitable for a sufferer of a food sensitivity. The mobile health monitor includes a reader that reads the barcode of the food product, a database having information about a plurality of food products to determine the suitability of the respective food product, and an indicator that provides an indication when a food product is suitable for the suffer.

The database is searched based on the read barcode to determine if the food product associated with the barcode is suitable. The indicator indicates the suitability of food product based on the results of the searched database. The indicator may be visual and/or audible.

An interface operatively connected to the database is provided in order to update the database.

In another aspect, a method for determining when a food product is suitable for a sufferer of a food sensitivity is provided. The method includes scanning a barcode of the food product via a mobile health monitor, decoding the barcode by the health monitor, using the decoded barcode to search a database having information for a plurality of food products, the information for determining the suitability of the respective food product, and indicating the suitability of food product based on the search. The food product is unsuitable when the food product is not found during the search.

### BRIEF DESCRIPTION OF THE DRAWINGS

The above mentioned and other concepts of the present invention will now be described with reference to the drawings of the exemplary and preferred embodiments of the present invention. The illustrated embodiments are intended to illustrate, but not to limit the invention. The drawings contain the following figures, in which like numbers refer to like parts throughout the description and drawings wherein;

FIG. 1 illustrates a chart of common symbols used on food packaging according to the prior art;

FIG. 2 illustrates an exemplary schematic diagram of a mobile health scanner according to an embodiment of the present invention;

FIG. 3 illustrates an exemplary system diagram for a database update of the mobile health scanner according to an embodiment of the present invention; and

FIG. 4 illustrates an exemplary use of the mobile health scanner according to an embodiment of the present invention.

### DETAILED DESCRIPTION OF INVENTION

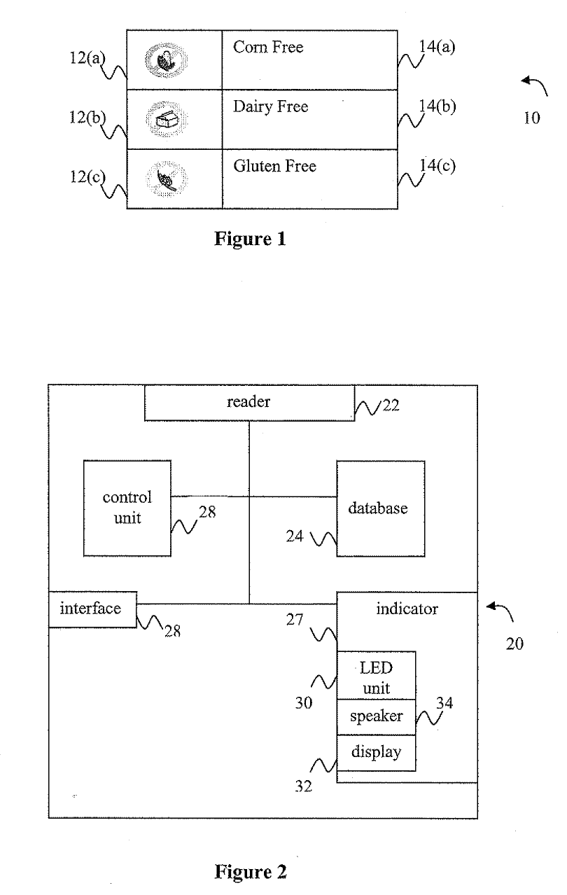

It is imperative for an individual with a food allergy not to consume the food item causing the sensitivity. This is especially important for individuals that have severe symptoms which are common in the cases of sensitivity to nuts, shellfish and gluten (celiac disease). However, food products are not always clearly marked of items known to cause sensitivity. In some cases the food product has a symbol indicating an item is excluded. FIG. 1 includes a chart 10 of a few common symbols 12 and their corresponding description 14. Unfortunately, this marking is voluntary. Furthermore, the marking is typically only done by manufactures of specialty foods, such as dietetic and organic products, which usually cost more than equivalent suitable products in a supermarket. Furthermore, this method may not be of use to the visually impaired.

Although food labeling rules have improved, they are still not adequate for identifying all food items that may cause sensitivity. For example, in the European Union the following items must be listed on the label.
cereals containing gluten (i.e. wheat, rye, barley, oats, spelt, kamut or crosses thereof),
crustaceans and crustacean products,
eggs and egg products,
fish and fish products,
peanuts and peanut products,
soy and soy products,
milk and dairy products (including lactose),
nuts (i.e. almonds, hazelnuts, walnuts, cashew nuts, pecan nuts, brazil nuts, pistachios, macadamia nuts) and products made therefrom,
celery and celery products,
mustard and mustard products,
sesame seeds and sesame seed products, and
sulfur dioxide and sulfites in concentration of more than 10 mg/kg or 10 mg/l, shown as SO₂.

There are exceptions to the labeling rules. For example:
if the item is already mentioned in the commercial description (e.g. peanuts in peanut butter), it does not need to be listed again in the list of ingredients,
for spice and or herb mixtures (e.g., curry or herbs from Provence) and for items governed by EU law such as chocolate or jams, a 2% rule applies such that only items that make up more than 2% by weight of the finished product may need to be listed,
for some packaged foodstuffs such as honey no list of ingredients is necessary, and
the items of unpackaged food do not need to be declared.

Furthermore, some manufactures include broad disclaiming statements such as “may contain traces of peanuts”. This is commonly done since the manufacture cannot always control items from different suppliers. However these widespread over-cautious declarations are not necessarily helpful to an individual suffering from food sensitivity.

Books may also be used to identify food products that an individual with food sensitivity may eat. However, the books are typically geared toward a single food item, for example, the German Celiac Disease Society publishes a list of gluten-free foodstuff in a book each year. Thus an individual suffering from multiple sensitivities would have to reference multiple books. Also, the book may not be available to everyone or at least not available when shopping for the food products. Furthermore, the book must be republished in order to be current. Meanwhile new food products that the sufferer may eat may have been introduced to the market.

There is a need for an easier way for an individual having food sensitivity to identify food products which are safe to eat.

Manufactures currently place barcodes on products to identify certain aspects of the product. For example, the 13 digit European Article Number (EAN), which is a super set of the 12-digit Universal Product Code (UPC) may be broken down as follows:

| | |
|---|---|
| Digits 1-3: | 3-digit country code, |
| Digits 4-7: | 4-digit manufacturer code, |
| Digits 8-12: | 5-digit product code generated by the manufacture, |
| Digit 13: | 1-digit check digit which is a checksum value to check for |
| | errors in the barcode. |

An individual may then scan the bar code using a mobile health scanner to determine if a food product is suitable or unsuitable for a person suffering from a food sensitivity. A suitable food product would not include any food items causing food sensitivity to the sufferer. An unsuitable food product includes at least one food item causing food sensitivity to the sufferer. An indeterminate food product is a food product that cannot be positively determined as a suitable or unsuitable for the sufferer. An indeterminate food product should be considered unsuitable

Referring to FIG. 2, an exemplary embodiment of the mobile health scanner 20 is provided. The mobile health scanner 20 includes a bar code reader 22, an integrated database 24, an interface 26, an indicator 27, and a control unit 28.

Bar code readers 22, which are also known as bar code scanners, are well known for reading printed barcodes. Types of bar code readers 22 may include a pen type reader, laser scanners, charge-coupled device (CCD) reader, which are also known as light-emitting diode (LED) scanner, and camera-based readers.

The integrated database 24 provides information in order to determine the suitability or unsuitability of food product. The integrated database 24 may be accessed using the manufacture code and product code and possibly the country code of the barcode. Thus, barcodes having a manufacturer code other than EAN, for example UPC, may be used.

In order to determine the suitability of the food product, the integrated database 24 may provide information such as the food items included in the food product. Thus, the suitability may be determined by checking the food item list for food items that cause food sensitivity. Alternatively or in addition to the food item list, a status of the food product to indicate the suitability of food product may be stored in the integrated database 24. A single status such as suitable or unsuitable could be used or any combination of suitable, unsuitable and indeterminate may be used.

The interface 26 is provided to connect to a computer at least in order to update the integrated database 24. The interface 26 may provide a wired and/or wireless connection to the computer. For example, the interface 26 may have a Universal Serial Bus (USB) port for connecting to the computer using a USB connector and/or antennae for a wireless connection via such standards as Wi-Fi or Bluetooth.

A means to indicate to the user information about the food product relating to food sensitivity is provided via an indicator 27. The indicator 27 may include for example a display 32, an LED unit 30, a speaker 34 or combinations thereof.

The display 32 visually provides information to the individual, for example, a list of food items may be displayed and possibly scrolled through. The display might only show specific food item(s) or the food item(s) may be highlighted within the list of food items.

The LED unit 30 provides visual indication of the suitability of the food product for the sufferer. The LED unit 30 includes at least one LED to indicate the food product is suitable or unsuitable. However, more LEDs may be provided. For example, a first light could indicate that the food product is suitable and a second light could indicate that the food product is unsuitable or indeterminate. However, a third light could be used to separate the unsuitable and indeterminate displays. When the LED unit 30 includes multiple LEDs, different color LEDs may be used for each indicator. For example a green LED could indicate a suitable food product whereas a red LED could indicate an unsuitable food product.

A speaker 34 would include an audible indication of the suitability of the food product. This is especially useful for the visually impaired. At least one sound would be needed to indicate if the product is suitable or unsuitable. However, multiple sounds could be used similarly to the LED indications. For example, different sounds could have different meanings such a one sound for a suitable product and another sound for an unsuitable product. A sound may be any audible indication such as a tone or voice command.

The control unit 28 is any circuitry and/or CPU to operate and/or operatively connect the barcode reader 22, the integrated database 24, the interface 26 and the indicator 27.

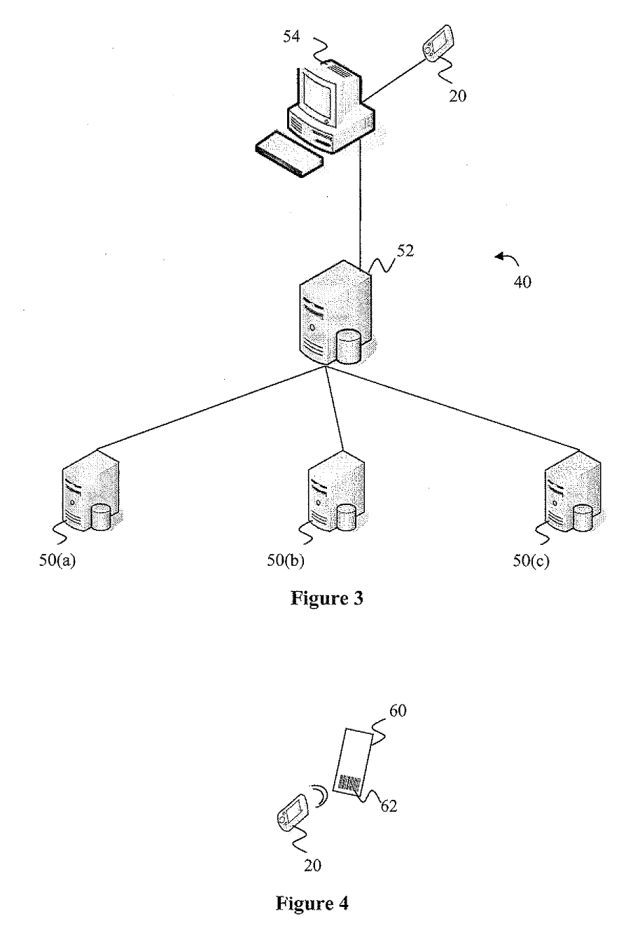

Referring now to FIGS. 2 and 3, an exemplary system 40 for a database update of the mobile health scanner is illustrated. The system 40 includes a host computer center 52 on the internet and a user computer 54 which is communicatively coupled to the host computer center 52. The host computer center 52 includes information about food products such as the ingredient lists and/or indications about the suitability of a food product based on an ingredient or a food sensitivity type. The mobile health scanner 20 is communicatively coupled to the user computer 54. An update may initiated for example via the user computer 54, the mobile health scanner 20 or the internet, in which at least a part of the data on the host computer center is transferred to the mobile health scanner 20 via the interface 28

The host computer center 52 may also receive updates from further databases 50. The further database 50 may be provided by various sources such as a food manufacture or support group.

In the present example, statuses for only the food products that are suitable for the sufferer are downloaded and stored at the mobile health scanner 20. This reduces how much data needs to be downloaded and thus stored. Furthermore, if a status is not stored for a food product, the food product would then be considered unsuitable.

In order to download these selective statuses, information of the food item(s) and/or food sensitivity, such a celiac disease, would need to be provided to the host computer center 52. This information may be provided of download by the user or be stored for example in the mobile health scanner 20, the user computer 54 or other location such as the intemet. Once this information is provided to the host computer center 52, the host computer center 52 would only transfer the appropriate status.

In other embodiments, it would not be necessary to provide the food item and/or food sensitivity to the host computer center 52. The host computer center 52 could transfer all status and/or food items to the user computer 54. The user computer 54 may then filter the transferred information and transfer the filtered status to the mobile health scanner 20 or transfer the information without filtering. Filtering may also occur at the mobile health scanner 20.

Referring now to FIGS. 2 and 4, an exemplary method is provided for determining the suitability of a food product. The mobile health monitor 20 scans a barcode 62 of a food product 60 to determine the manufacture code and the product code, which is embedded in the barcode 62. The control unit 28 uses the manufacture and product codes, which are decoded from the barcode 62, to search the integrated database 24 for information stored about the food product associated with the manufacture and product codes. Based on the information found an indication may be provided to the user of the mobile health monitor 20 by the indicator 20.

In the present example, the database includes a status of the food product 60 which indicates that food product 60 is suitable. Based on the status, the control unit 28 turns on an LED, which is displayed on the LED unit 30, to indicate to the user that the food product 60 is suitable.

An alternative to a barcode would be the use of a Radio Frequency Identification (RFID). It is anticipated that RFID will replace barcodes. RFID tags may be imbued with intelligence, individuality and communicability. Thus, a central product database may no longer be needed.

While the invention has been described in terms of a certain preferred embodiment and suggested possible modifications thereto, other embodiments and modifications apparent to those of ordinary skill in the art are also within the scope of this invention without departure from the spirit and scope of this invention. Thus, the scope of the invention should be determined based upon the appended claims and their legal equivalents, rather than the specific embodiments described above.

1. A mobile health monitor system that indicates that a food product is suitable for a sufferer of a food sensitivity; comprising:
a mobile health monitor comprising a reader configured to read a barcode of the food product, a mobile health monitor database having information about a plurality of food products to determine the suitability of the food product, the database being searchable based on information obtained from reading the barcode, and an indicator configured to provide an indication to identify food product suitability; and
a remote processing unit configured for releasable connection to the mobile health monitor, the remote processing unit having a food suitability database comprised of food product information: and
wherein the mobile health monitor is configured to communicatively couple to the remote processing unit such that the mobile health monitor database is updated to include at least a portion of the food product information of the food suitability database.

2. The mobile health monitor system according to claim 1, wherein the indication is visual.

3. The mobile health monitor system according to claim 1, wherein the indicator is comprised of at least one LED.

4. The mobile health monitor system according to claim 1, wherein the indication is audible.

5. The mobile health monitor system according to claim 1, wherein only information for suitable food products is stored in the mobile health monitor database.

6. The mobile health monitor system according to claim 1 wherein the remote processin unit is a computer or a host computer center.

7. The mobile health monitor system according to claim 6, wherein only information for suitable food products is transferred to the mobile health monitor when the mobile health monitor database is updated.

8. A method for determining when a food product is suitable for a sufferer of a food sensitivity, comprising:
entering food sensitivity information into a mobile health monitor;
communicatively coupling a remote processing unit to the mobile health monitor to update a database of the mobile health monitor to include food product status information for food products based on food sensitivity information entered into the mobile health monitor:
reading a food product via a mobile health monitor;
determining food product information for the food product with the mobile health monitor;
searching the database of the mobile health monitor to determine the suitability of the food product:
indicating the suitability of the food product by an indication emitted or displayed by the mobile health monitor; and
wherein the food product is indicated as being unsuitable when information for the food product is not found during the searching of the database.

9. The method according to claim 8, wherein the database is searched by product code.

10. The method according to claim 8, wherein the remote processing unit is a host computer center or a computer.

11. The method according to claim 8, wherein only food product status information for food products is transferred to the database of the mobile health monitor when the database is updated.

12. A health monitor system for a sufferer of a food sensitivity comprising:
a mobile health monitor comprising a reader configured to read food product information from at least one food product packaging apparatus, an indicator configured to provide an indication, and a user specific mobile health monitor database having information about a plurality of food products, the mobile health monitor configured to search the user specific mobile health monitor database for user food product status information based on food product information obtained by the reader;
a host processing unit having a host database, the host processing unit releasably connected to the mobile health monitor, the host database having food product information and food ingredient information; and
wherein the mobile health monitor is configured to communicativeLy couple to the host processing unit such that the host database is searched based on at least one user food sensitivity ingredient and the user specific mobile health monitor database is updated with user food product status information for food products.

13. The system of claim 12 further comprising at least one third party processing unit configured to releasably connect to the host processing unit such that the food product information and food ingredient information is updated by the at least one third party processing unit.

14. The system a claim 13 wherein the at least one third party processing unit is a plurality of third party processing units, each third party processing unit controlled by a different food manufacturer.

15. The system of claim 12 wherein the reader is a bar code reader.

16. The system of claim 12 wherein the food product packaging apparatus is a bar code attached to packaging of a food product.

17. The system of claim 12 wherein the food product packaging apparatus is a radio frequency identification device emitting a radio frequency identification and the reader is configured to receive the radio frequency identification.

18. The system of claim 12 wherein the mobile health monitor database is updated only with user food product status information for food products that do not contain the at least one user food sensitivity ingredient.

19. The system of claim 12 wherein the mobile health monitor is configured to receive user food sensitivity information identifying the at least one user food sensitivity ingredient and the mobile health monitor is configured to send the user food sensitivity information to the host processing unit when communicatively coupled to the host processing unit such that the mobile health monitor database is updated only with user food product status information for food products that do not contain the at least one user food sensitivity ingredient.

20. The system of claim 12 wherein the host processing unit is configured to receive food sensitivity information that identifies the at least one user food sensitivity ingredient from a remote user processing unit releasably connected to the host processing unit via a communication network selected from the group consisting of wireless networks, interconnected remote processing units, active networking networks, client-server networks, peer-to-peer networks, internetworks, global area networks, and virtual private networks.
